(19) United States
(12) Patent Application Publication
(10) Pub. No.: US 2021/0121296 A1
(43) Pub. Date: Apr. 29, 2021

(54) RADIAL-CAPITELLAR IMPLANT

(71) Applicant: OT MEDIZINTECHNIK GMBH, München (DE)

(72) Inventor: Ulrich SCHREIBER, München (DE)

(21) Appl. No.: 16/618,310

(22) PCT Filed: Jun. 4, 2018
(86) PCT No.: PCT/EP2018/064663

§ 371 Date: Nov. 29, 2019

(30) Foreign Application Priority Data
Jun. 2, 2017 (DE) 10 2017 112 244.3
Nov. 13, 2017 (DE) 10 2017 126 618.6

(51) Int. Cl. A61F 2/38; A61B 17/15; A61F 2/46

(52) U.S. Cl. CPC A61F 2/3804; A61B 17/15; A61F 2002/30367; A61F 2002/3827; A61F 2/4605

(57) ABSTRACT

The present invention relates to a radial head implant with a head, a shaft and a threaded anchor.

The present invention relates to a radial head implant and a head therefor, a shaft, a washer, a threaded anchor of a radial head implant and to a sawing template, a head-applying tool a shaft-implanting tool, a bone processing tool, a ratchet and to a method for inserting the radial head implant as well as to a kit.

A traumatic fracture, a lesion or an aberration of a radial head often requires a synthetic replacement in the sense of a radial head implant. Such an implant is subject to many requirements. It is, amongst others, desirable to (be able to) insert an implant in a minimally invasive manner.

It is the object of the present invention to provide a head for the radial head implant, a threaded anchor for a radial head implant, a shaft for a radial head implant, a washer for a radial head implant, a further radial head implant, a sawing template, a head-applying tool, a shaft-implanting tool, a ratchet, a bone processing tool, a kit and a further method for inserting a radial head implant.

The object according to the present invention is achieved by the head for a radial head implant according to claim 1, the threaded anchor for a radial head implant according to claim 5, the shaft for a radial head implant according to claim 7, the washer for a radial head implant according to claim 15, the sawing template according to claim 16, the radial head implant according to claim 16, the head-applying tool according to claim 17, the shaft-implanting tool according to claim 18, the ratchet according to claim 19, the bone processing tool 20, the kit according to claim 21 and the method for inserting a radial head implant according to claim 22.

In all of the following statements, the use of the expression “may be” or “may have” and so on, is to be understood synonymously with “preferably is” or “preferably has,” and so on respectively, and is intended to illustrate embodiments according to the present invention.

Whenever numerical words are mentioned herein, the person skilled in the art shall recognize or understand them as indications of numerical lower limits. Hence, unless it leads to a contradiction evident for the person skilled in the art, the person skilled in the art shall comprehend for example “one” as encompassing “at least one”. This understanding is also equally encompassed by the present invention as the interpretation that a numerical word, for example, “one” may alternatively mean “exactly one”, wherever this is evidently technically possible in the view of the person skilled in the art. Both of these understandings are encompassed by the present invention and apply herein to all numerical words used herein.

Unless otherwise stated below, proximal means towards the body center and distal means away from the body center. With regard to the radial head implant, proximal and distal are to be understood with respect to the position of the radial head implant when implanted as intended.

The head for a radial head implant according to the present invention comprises a, in particular rotatable, interlocking element for fastening, in particular releasably fastening, the head. Preferably, the interlocking element serves for fastening the head on a shaft of the radial head implant. The interlocking element is preferably a separate component that can be inserted into the head.

Fastening the head on the shaft in the sense of the present invention means in some embodiments that the head is connected to the shaft, wherein, however, relative movements in one or more directions or planes are allowed, for example, a rotation of the head around the longitudinal axis of the shaft is still possible; in other embodiments, fastening means a fixing or fixation which also prevents a rotation of the head.

The shaft for a radial head implant according to the present invention comprises an upper shaft part and a lower shaft part. In this, the upper shaft part is designed to be fastened on the head of the radial head implant; in other words, the head may be fastened on the upper shaft part. In this, the upper shaft part may, for example, substantially have the form of a dish or plate. The lower shaft part is designed to be inserted into a bone, in particular into the medullary cavity of a radial shaft. The lower shaft part comprises, for this purpose, preferably an elongated shape, which is preferably substantially or partially cylindrical.

The threaded anchor for a radial head implant according to the present invention comprises a—preferably self-tapping—thread and at least one, in particular central or decentral through-opening.

In several embodiments, the thread is designed to establish and ensure the primary stability of the radial head implant.

In several embodiments, the threaded anchor is designed at its outwardly directed portion such that osseointegration is promoted, for example, by macro- or micro-structuring and/or by a coating. However, in some embodiments, the inwardly directed portion (the central through-opening) is smooth (e.g. polished, coated and/or hardened), so that this portion may slide relative to the shaft, has low frictional forces, minimizes abrasion and/or inhibits osseointegration.

In several embodiments, the threaded anchor is equipped at its outwardly directed portion with a rougher surface than at its inwardly directed portion (the central through-opening). This may be optionally the case regardless of the thread.

The washer for a radial head implant according to the present invention is designed to be inserted between upper shaft part and bone, in particular between the distal surface of the upper shaft part and radial shaft. The washer serves preferably for adjusting the desired distance between the radial shaft (after resection of the bony radial head) and the proximal side of the head. It thus serves for adjusting the joint's tension (to avoid the phenomenon that is referred to as over-/under-stuffing).

The radial head implant according to the present invention comprises a head (which optionally comprises an interlocking element), a threaded anchor and a shaft. These are each preferably provided separately from each other or are separable from each other, in particular without requiring tools or without to be destructive.

The radial head implant according to the present invention is preferably suited to be used in a minimally invasive procedure. For this purpose, after a resection of the radial head, the shaft with positioned threaded anchor may preferably be pivoted into the radius which is advantageously possible even in very small resection cavities.

The sawing template according to the present invention comprises a plate with a tongue which is mounted completely or at least partially parallel to the plate, wherein the tongue comprises preferably a convexity towards the plate. In some embodiments, instead of the plate, the sawing template may also comprise a guide configured for an oscillating saw, for example a slot or an opening.

The head-applying tool according to the present invention is designed and provided for receiving the head according to the present invention and for fastening the head according to the present invention on a shaft being introduced into a radius.

The shaft-implanting tool according to the present invention is in particular designed and provided for inserting the shaft with the threaded anchor or another anchor into the medullary cavity of a radius. In several embodiments, the shaft-implanting tool may also be used to insert the shaft without an anchor, in particular without a threaded anchor, into the bone, in particular when the shaft is inserted with bone cement and without anchor.

The bone processing tool according to the present invention comprises a stalk and a rasping element arranged laterally on the stalk.

The ratchet according to the present invention is designed for screwing a threaded anchor into a radius by a rotation of the shaft.

Preferably, the ratchet may be used for explantation with a reverse freewheel. Alternatively, the ratchet may in some cases be used reversed, e.g. turned around, wherein the freewheel is not reversed.

The kit according to the present invention encompasses the radial head implant according to the present invention and at least one of the following aids according to the present invention:
sawing template;
head-applying tool;
shaft-implanting tool;
ratchet; and/or
bone processing tool.

The method for inserting a radial head implant according to the present invention encompasses at least a plurality (i.e. two or more) of the following steps:
mounting and/or aligning a sawing template at a radial head, at a capitulum humeri and/or at its joint space and mounting and/or aligning the sawing template along a radial shaft;
determining the sizes of the radial shaft and/or of the radial head;
resecting the radial head along a side of the plate or guide of the sawing template being preferably according to the present invention, said side being distal to the radial head;
expanding the medullary cavity of the radius using a bone processing tool being preferably according to the present invention;
introducing a shaft, being preferably according to the present invention, comprising a threaded anchor into the medullary cavity of the radius using a shaft-implanting tool;
screwing the shaft, being preferably according to the present invention, comprising the threaded anchor into the radius using a ratchet;
placing a head, being preferably according to the present invention, on the shaft using a head-applying tool;
interlocking an interlocking element of the head, being preferably according to the present invention, using the head-applying tool; and
optionally: introducing a washer being preferably according to the present invention.

Advantageous developments of the present invention are each subject-matter of the dependent claims and embodiments.

Whenever an embodiment is mentioned herein, it is then an exemplary embodiment according to the present invention, which is not to be understood as limiting.

Embodiments according to the present invention may comprise one or several of the features mentioned supra and/or in the following in any combination unless the person skilled in the art recognizes such a combination to be technically impossible.

The interlocking element is preferably designed rotatable. Alternatively, the interlocking element may also be designed as in introduction cascade, which may optionally also be closable.

The optionally rotatable interlocking element of the head is preferably designed for fastening the head on the shaft. For this purpose, the interlocking element comprises in several embodiment a latch with which part of the shaft may be interlocked in the head. he interlocking element may be inserted in an opening of the head, in particular in a blind hole. Preferably, the interlocking element is designed such that its surface directed outwardly is flushed with the head contour, in a closed rotation position.

In several embodiments, the latch has optionally the form of a key bit. The optionally rotatable interlocking element may interlock the head to the shaft, in that the latch hooks optionally form-fit on the shaft by a rotation of the interlocking element. This may ensure that the interlocking element is on one hand secured against pulling it out of the head and on the other hand, that the head is fixed to the shaft preferably in a form-fit connection.

The interlocking element is preferably designed such that a turning of the interlocking element may result in a releasable fastening and/or interlocking of the head at the shaft.

The interlocking element comprises preferably a securing element which interacts with the head such that the interlocking element is snapped-in or clamped in the head.

In several embodiments, the securing element is not a force-fit securing element, no clamping screw, no fixing screw and/or no locking pin.

In several embodiments, the interlocking element comprises no T-profile. A T-profile may be designed for the form-fit connection of the shaft with the head.

In several embodiments, the interlocking element comprises no pin or fastening pin. The pin or the fastening pin may be designed for the form-fit securing of the head on or at the shaft.

In several embodiments, the rotatable interlocking element comprises at least one detent element for at least one rotational position.

In this, the detent element may in some cases serve for allowing the interlocking element to latch or snap-in in one or several rotation position(s). For this purpose, the head is preferably designed to interact with the detent element of the interlocking element. For example, the interlocking element may comprise, as an example of a detent element, at least one detent nose, which may latch in at least one detent depression in the head, in particular in the opening of the head.

In several embodiments, the interlocking element may latch in an open rotational position in which the shaft may be released from the head and/or in a closed rotational position in which the head is fastened at the shaft.

In several embodiments, the interlocking element comprises a drive. The drive is a device at which a tool may be positioned and may transmit a torque to the interlocking element, in order to turn the interlocking element or to actuate it differently. In this way, the interlocking element may be, for example, rotated or turned (or moved) from an open position to a closed position and vice versa.

The interlocking element may be transferred from an open position to a closed position and vice versa preferably by a rotation of about 90°.

In several embodiments, a drive encompasses a hexagon socket and/or a slot, in each of which a rotation tool may engage.

In several embodiments, the head is designed such that when in a functional position, it is in contact with the corresponding capitulum humeri or with the replacement of a capitulum humeri. In this, the head comprises in several embodiments a smooth or very smooth surface and/or no sharp edges. In addition or alternatively, the material of the head is preferably designed such that there is a hard-soft-matching between head and capitulum humeri. Alternatively, the material of the head may be designed such that there is a hard-hard matching, for example, by ceramic surfaces.

In several embodiments, the head comprises a concave curvature or plate curvature on the side facing the capitulum humeri in the functional position.

In several embodiments, a plurality, preferably five, different head sizes are provided when applying the radial head implant. In this, the different head sizes preferably comprise a proportional plate curvature and plate depth. In this, the outer contour of the head is adapted for an advantageous radio-ulnar joint effect. Preferably, the concave curvature and the diameter of the head are adapted for an advantageous radio-capitular articulation and/or radio-ulnar articulation. In addition, the head is preferably designed such that the articulation between the head and the medial side of the trochlea advantageously leads to a stabilization.

In several embodiments, the shaft and/or the threaded anchor is also provided in a plurality of sizes, in particular five sizes. In this, preferably all shaft sizes may be combined with all head sizes.

In several embodiments, the head may be fastened and/or interlocked at the shaft such that movement of the head is possible, particularly a relative movement between the head and shaft, particularly a rotation about the longitudinal axis of the shaft, compensating for any incongruity present between articulation surface of the head and capitulum humeri. With free rotation of the head, the translational movement of the head perpendicular to the longitudinal axis of the lower shaft part and/or along this longitudinal axis may in this be free or hindered.

In other embodiments, the head may be fastened at the shaft such that a movement, in particular a rotation, of the head is prevented. When the rotation of the head is prevented, the translational movement of the head perpendicular to the longitudinal axis of the lower shaft part and/or along this longitudinal axis may in this be free or hindered.

In several embodiments, the proximal contour of the head is rotationally symmetric and comprises a concave surface. In this, the radius of this concave contour does in some cases not correspond to the capitulum humeri of the convex curvature of the capitulum humeri in every angular position of the head. In addition, in some cases, the maximum of the capitulum humeri may shift or wander within a movement relative to the minimum of the head.

In several embodiments, the head comprises an opening at its lower side, which, in use position of functional position, faces away from the capitulum humeri. This opening possesses in an extension direction at least two different dimensions. Preferably, at least two different dimensions of the opening exist in an extension direction at right angles or across of the main extension direction of the opening. In several embodiments, the opening is disposed laterally on the head.

In several embodiments, the opening at the lower side of the head comprises a narrow portion and a wider portion. In several embodiments, the opening comprises the contour of a keyhole. The contour of a keyhole consists in some cases of one (more or less complete) circle, to which a rectangle directly connects with its short side. In this, the (possibly fictitious) diameter of the circle is larger than the length of the shorter side of the rectangle.

In several embodiments, the threaded anchor for a radial head implant comprises a self-tapping thread to be screwed into the cortical and/or cancellous bone. The shaft is preferably movable within the threaded anchor, in particular in an axial direction of the lower shaft part. Preferably, the shaft cannot be rotated relative to the threaded anchor.

The threaded anchor comprises preferably a trapezoidal thread, which is particularly preferably designed self-locking in order to counteract a loosening.

The thread of the threaded anchor is in several embodiments self-tapping even in left-hand rotation. This may in some cases facilitate explantation.

Single degrees of freedom may selectively remain free to a defined extent at the interfaces between the components (head, threaded anchor and/or shaft) in order to allow a physiological adjustment to the joint surfaces of radius, ulna, capitulum humeri and/or trochlea. In several embodiments, the radial head implant possesses two degrees of freedom: the head is rotatable about the z-axis (longitudinal axis of the lower shaft part) and the shaft is movable along the z-axis and/or across the z-axis. In other embodiments, the radial head implant possesses only one of the two mentioned degrees of freedom. In several embodiments there is no degree of freedom: head and shaft are fixed. In several embodiments, the head is movable about +/−1 mm along the x-axis and the y-axis, so that there are two degrees of freedom.

In several embodiments, the threaded anchor allows a fixing on the isthmus of the medullary cavity.

In several embodiments, the threaded anchor tapers in distal direction, i.e. towards the end which is to be inserted first into the bone. In several embodiments, the basic shape of the threaded anchor represents a truncated cone, in which a through-opening is coaxially introduced and on the lateral surface of which a self-tapping thread is applied. In this, the thread is preferably periodically interrupted, e.g. two, three or four times along the circular circumference in order to carry away the removed bone material.

In several embodiments, the through-opening of the threaded anchor comprises at least one transmission element for transmitting a torque from an inserted lower shaft part to the threaded anchor. In this, such a transmission element encompasses preferably one, two or more grooves, notches, projections, teeth, protrusions, webs, noses or ridges. The shaft comprises preferably guide devices which cooperate with the transmission elements of the threaded anchor to transmit a torque from the lower shaft part to the threaded anchor. For this purpose, the lower shaft part comprises for example one or several groove(s), in which the nose(s) of the threaded anchor may engage in order to transmit a torque from the shaft to the threaded anchor. At the same time, the one or the several transmitting elements of the threaded anchor may preferably represent with the guiding device(s) a linear guide, so that the shaft remains movable in the threaded anchor along the longitudinal axis of the lower shaft part after the shaft has been screwed into the radius by the threaded anchor.

The shaft according to the present invention for a radial head implant comprises a lower shaft part which in several embodiments is designed to be inserted into the through-opening of the threaded anchor. In this, the lower shaft part is preferably provided with guiding elements which cooperate with transmitting elements of the threaded anchor, such that a torque may be transmitted from the shaft to the threaded anchor.

In several embodiments, the threaded anchor and/or the shaft comprise elements for the releasable fixing of the threaded anchor at the shaft, so that there results preferably a securing element of the shaft relative to the threaded anchor. In this, the threaded anchor may in some cases be clamped on the shaft and/or latched or snapped-in there. For example, at the transition between lower shaft part and upper shaft part, the lower shaft part may have a large diameter so that the threaded anchor may be clamped and/or latched. Thus, a slippage of the threaded anchor off the shaft during the operative implantation of the radial head implant is prevented. In some cases, the shaft is provided with an already applied threaded anchor captively secured.

In several embodiments, the shaft comprises, at the transition from the lower shaft part to the upper shaft part, at least one, optionally circumferential, groove. The groove may for example be part of the securing element, wherein for example portions of the threaded anchor may snap-in in the groove. The groove may alternatively or additionally serve for fastening washers to the shaft.

Instead of a groove, one or several elevation(s) may be provided at the shaft, which in turn may snap-in in one or several groove(s) of the threaded anchor.

In several embodiments, the upper shaft part comprises a holding pin. The holding pin is preferably attached to the side of the shaft facing the head. In this, the holding pin has preferably a smaller cross-section at its section facing the upper shaft part than at its section arranged further away from the upper shaft part. In several embodiments, the holding pin has the form of a mushroom head.

Preferably, the upper shaft part, after implantation, rests flush on the radial shaft when the radial head was resected. In this, the upper shaft part may preferably have an osseointegrative surface on the side directed towards the radial bone. The osseointegrative surface is preferably rough and/or has a macrostructure and/or coating. The other side of the upper shaft part, proximally directed, preferably has a smooth, in particular hardened and/or polished and/or coated surface, which preferably generates little friction and/or abrasion in the articulation with the radial head and/or restricts or prevents a relative movement by a force-fit connection between head and upper shaft part.

Due to an upper shaft part resting in a flush manner during use as intended, the primary alignment of the shaft and head is preferably determined by the resection orientation.

In several embodiments, the holding pin may be inserted into the opening of the lower side of the head. In this, the wider portion of the holding pin may preferably be inserted in a wider portion of the opening at the head, but preferably not in a narrower portion of the opening. By shifting the holding pin after the insertion into the opening in direction of the narrower portion of the opening, the holding pin may preferably no longer be axially removed from the opening. In several embodiments, the holding pin is a mushroom head and the opening has a keyhole shape, in this, the mushroom head may be inserted through the round portion of the keyhole and may then be shifted to the narrower portion of the keyhole, taking advantage of the narrower stem of the mushroom head; after the shifting, the mushroom head can preferably no longer be axially removed from the keyhole.

In several embodiments, the interlocking element of the head is designed to allow, in an open position, the holding pin to be inserted into the opening of the head and to prevent, in a closed position, the removal of the holding pin from the head. In several embodiments, this is realized by the interlocking element preventing a shifting of the holding pin, which is inserted in the opening, along the opening, for example by a latch of the interlocking element.

Using the latch, for example there may be generated a form-fit connection between head (in particular the interlocking element) and holding pin.

In several embodiments, the shaft which may be cylindrical or conical or differently tapered in any embodiment, may also be inserted into the medullary cavity also without a threaded anchor and may be anchored there by bone cement.

The shaft is used during implantation to be advanced into the medullary cavity of the radius. In this, in some embodiments, even after screwing the threaded anchor into the radius, a relative movement between threaded anchor and shaft may be temporarily or permanently possible.

The upper shaft part comprises, measured from a longitudinal axis of the lower shaft part, an extension in a first direction, which differs from an extension in a second direction. The upper shaft part may for example comprise a limitation which represents a circular arc whose ends are connected by or with a straight line. In several embodiments, the upper shaft part is designed particularly flat and/or has a rounded contour, which may facilitate pivoting the shaft into the radius, especially in a minimally invasive procedure.

Preferably, the material of the shaft comprises at least on the following characteristics: sufficient ductility, minimal abrasion, hard contact surfaces, smooth contact surfaces.

In several embodiments of the head, of the shaft, of the threaded anchor and/or of the washer, each of them is, due to its material or to an additional coating, antibacterial and/or comprises a structured and/or a coated osseointegrative area or section.

The sawing template serves for the precise resection of the radial head. The sawing template is preferably available in several sizes. The sawing template comprises a plate, in particular a sawing plate, in particular for guiding an oscillating saw (in particular with an oscillating saw blade with an end-face cutting surface). Instead of the plate, the sawing template may also be equipped with a differently shaped guide, in particular an opening for guiding an oscillating saw. In this, the plate has preferably a contour in order to apply the plate to the radius, in particular to the radial shaft. For this purpose, the plate comprises preferably an arc which may be applied to the radial head. By the arc, the radial shaft or the radial head may be contacted which improves the guiding of the saw and its stabilization during the cutting process and may act as size determination.

In addition, the sawing template comprises a tongue, which may be additionally or alternatively applied at the capitulum humeri. In several embodiments, the contour of the plate, follows in parts a circular arc. The tongue is preferably attached via a connecting element parallel to the plate. The connecting element is preferably attached to tongue and plate in a right angle. The tongue comprises, preferably in a portion, a convexity (in particular in shape of a spoon) towards the plate, i.e. the tongue bulges in the direction of the plate. In this, the form of the tongue may serve for the size assignment and/or alignment of the sawing template. The sawing template may be applied on a radial bone such that the arc of the plate rests laterally on the radial head or on the radial shaft and the tongue is disposed the joint space between radial head and humerus, wherein the tongue with it convexity preferably contacts or rests on the concave surface of the radial head. The connecting element is preferably provided with at least one borehole or one through-opening which preferably does not extend in the right angle to the connecting element. Herethrough, preferably wires (in particular K-wire) may be pushed for fixing the sawing template, e.g. on the humerus.

In an embodiment of the sawing template, which may be used both left and right, at least two openings are arranged or attached at the sawing template, in particular at the connecting element, preferably one for use on the left and one further for use on the right arm. In this, the openings are preferably marked according to their use, e.g. with R and L.

The sawing template may also serve, in several embodiments, to determine the size of the radial head to be replaced. For this purpose, there are preferably several sawing templates with different sizes provided in a kit, which differ for example with regard to the radius of the arc of the plate and/or in the size of the tongue (in particular its convexity). Additionally or alternatively, the sawing templates may differ in their extension in longitudinal direction in order to show for example the depth of the trochlea.

After placing the corresponding sawing template, an oscillating saw may be guided along the distal side of the plate (relative to the radial head), so that the radius or the radial head may be resected in a defined plane. For this purpose, a part of the plate may be used as a grip for handling the plate.

In several embodiments, the sawing template may be used in conjunction with a size gauge. In this, the plate may comprise a guide for a slide of the size gauge. When the tongue is inserted in the joint space, the gauge may be shifted along the guide to the radial head so that the radial head touches an arm of the size gauge. The arm is deflected by the radial head so that a pointer connected to the arm indicates the size of the radial head on a scale. The portions according to the present invention of the radial head implant as well as the associated tools may be selected on the basis of the values read from the scale.

In some cases, as a result, resection may advantageously be carried out which provides optimum precondition for a subsequent implantation of a radial head implant.

A template having a plurality of circular recesses and which may be associated with a kit may be used to check the size of the radial head after removal. For this purpose, the resected radial head is inserted into the recesses and checked whether it fits to matches with the measurements of the recesses. The recesses are preferably marked in order to select the suitable radial head implant.

The bone processing tool according to the present invention comprises preferably a flat stalk on which a rasping element is attached on a side towards one end. The rasping element extends conically away from the stalk preferably at a right angle. In several embodiments, the rasping element has substantially the shape of a truncated cone. The surface of the rasping element is preferably designed like the surface of a rasp or file, i.e. it comprises for example notches or teeth.

After the resection of the radial head, the bone processing tool with the rasping element may be inserted into the medullar cavity of the radius. By rotating the bone processing tool, the medullar cavity may be extended by cutting. The rasping element comprises preferably a design height that allows a minimally invasive introduction into the processing cavity. In several embodiments, the bone processing tool may have a ratcheting function.

Following a sufficient enlargement of the medullary cavity using the bone processing tool, the shaft may be inserted with the threaded anchor into the radius. Sufficient means here that the diameter of the widened medullary cavity preferably corresponds to the core diameter of the threaded anchor.

The form of the rasp and the procedure of widening the bone support (the radial shaft) is preferably designed such that the shaft is not burst and the bone support is not mechanically stressed excessively. Also, the subsequent implantation of the shaft and the screwing of the threaded anchor are preferably done so that the bone support is not overstressed.

In several embodiments, the bone processing tool may be made of two parts. In this, a first part preferably comprises a stalk, a grip and/or a ratchet for manipulation (in particular for a rotary or pivoting movement about the z-axis of the radial shaft) of the, in particular interchangeable, second part, in particular a rasping element. In this, the size of the rasp may preferably be used stepwise from small to large. In this, the rasping element is movable in z-direction of the stalk. This may make it possible that the stalk always rests flat on the resection surface of the radial shaft and thus the rasping direction is determined perpendicular thereto.

To insert the shaft with the applied threaded anchor into the radius, the shaft-implanting tool may be used. The shaft-implanting tool encompasses preferably the holding pin of the upper shaft part, in particular at the narrower point of the holding pin. Additionally, in several embodiments, the shaft-implanting tool may engage with one or several pin(s) in one or several hole(s) of the upper shaft part. The shaft-implanting tool comprises preferably an elastic element, in particular a spring, in order to fix the holding pin at the shaft-implanting tool. In several embodiments, the shaft-implanting tool is or comprises forceps whose forceps surface is shaped to embrace the holding pin of the shaft.

In some cases, the shaft with the attached threaded anchor may be inserted as follows: The upper shaft part is first placed with its distal side on the resection surface of the radial shaft. This may result in an anatomically correct orientation of the implant during and after screwing and an implant corresponding to the resection surface (xy-plane). Only by pushing down pins of the shaft-implanting tool, that is, by releasing a securing element of the shaft-implanting tool, on the threaded anchor, the threaded anchor grips in the bone. A subsequent rotational movement (in the direction of the thread) and the preferably self-tapping shape of the threaded anchor cause the distal incision and thus lead to anchoring the threaded anchor in the bone (so-called primary stability).

After inserting the shaft with threaded anchor using the shaft-implanting tool, the threaded anchor may be screwed into the radius. For this purpose, the ratchet may be used. The ratchet preferably comprises elements which may transmit a torque to elements of the upper shaft part. Preferably, the upper shaft part comprises one or several openings into which one or several pins of the ratchet may engage. In one embodiment, the ratchet comprises at least one or exactly one, two or several pins, respectively, and/or the upper shaft part comprises at least one or exactly one, two or several openings, respectively. By actuating the ratchet, a torque may be transmitted from the ratchet to the shaft and the threaded anchor, so that the threaded anchor is screwed into the radius. Preferably, the ratchet comprises a receptacle for the holding pin of the shaft. In several embodiments, the at least one, two or several pins and/or the receptacle of the ratchet are available on an attachment of the rachet. In this, the attachment may optionally be replaceable and in particular be provided in different sizes. The sizes of the attachment are preferably adapted to different shaft sizes.

In several embodiments, inserting the pin(s) of the ratchet into the shaft may cause the securing element of the threaded anchor to disengage.

A stalk of the ratchet preferably has a width of less than 15 mm, more preferably less than 12 mm, more preferably less than 10 mm and most preferably less than 9 mm. Preferably, the stalk of the ratchet is at least 5 mm, more preferably at least 7 mm, and more preferably at least 8 mm wide.

Preferably, the thickness of the stalk of the ratchet is less than 6 mm, more preferably less than 5 mm, more preferably less than 4 mm and most preferably less than 3 mm. In this, the thickness of the stalk of the ratchet is preferably at least 1.5 mm, more preferably at least 2 mm.

The ratio of width to thickness of the stalk of the ratchet is preferably at least 4, more preferably at least 6, more preferably at least 8 and most preferably at least 9. In this, said ratio is preferably less than 15, more preferably less than 12, and most preferably less than 10.

The said measurements for of thickness and width of the stalk of the ratchet are preferably in a range between 1 cm and 8 cm from the point where the ratchet is placed on the shaft, in particular measured from where the holding pin engages in the ratchet or its attachment. Particularly preferably, this range is between 2 and 6 cm, most preferably between 3 and 5 cm of said point.

In several embodiments, the shaft, after being screwed into the radius, remains axially (with respect to the longitudinal axis of the lower shaft part) movable in the threaded anchor, in particular axially displaceable.

Subsequently, the head can be placed on the shaft using a head-applying tool. In this case, preferably, the holding pin of the upper shaft part is inserted into the opening in the head. By a rotation of the head-applying tool, which leads to a rotation of the interlocking element of the head, the holding pin is interlocked in the head and therefore the head is fastened to the shaft. Preferably, a quarter turn is sufficient to transfer an open rotational position of the interlocking element in a closed rotational position.

In order to adjust the height (in an axial direction) of the joint space, the washer may optionally be fastened between the upper shaft part and the resection surface of the radius on the lower shaft part.

The washer is preferably designed such that after screwing in the threaded anchor using the shaft it may be pushed laterally on the lower shaft part. For this purpose, the washer comprises preferably a central opening and a recess which continues from the opening to a side until the edge of the washer. In this, the recess preferably has a smaller diameter than the lower shaft part at the transition to the upper shaft part, so that the washer has to be slightly elastically deformed, in particular tensioned, in order to be able to push it on the lower shaft part. In order to facilitate such deformation, the washer may have openings. Once the washer has reached its destination on the lower shaft part and the central opening of the washer embraces the lower shaft part, the washer relaxes so that the washer cannot slip off the lower shaft part.

The side of the washer facing the bone support may, in some cases, be designed osseointegrative due to its surface structure (e.g. by its microstructure and/or macrostructure, a coating, etc.) and, for example, be smooth on the side facing the shaft.

In several embodiments, the radial head implant comprises surfaces for a secondary anchoring. For this purpose, the radial head implant is at last partially provided with a surface for the secondary anchoring by ingrowth. In several embodiments, the radial head implant may comprise specific sites for the Osseointegration at at least one of the following locations: bottom of the upper shaft part, threaded anchor, tip of the lower shaft part, washer.

In several embodiments, the radial head implant has no surface for a secondary anchoring. Thus, the threaded anchor may be anchored primarily by screwing into the radius, wherein the shaft may remain to be movable along its longitudinal axis.

In several embodiments, there is a surface for the secondary anchoring on the threaded anchor. In this embodiment, the threaded anchor may grow in wherein the shaft, however, remains to be movable or shiftable along its longitudinal axis relative to the threaded anchor.

In several embodiments, the radial head implant comprises a surface for the secondary anchoring on the shaft or at least on parts of the shaft. Here, a secondary anchoring may take place via the shaft. After ingrowth, the shaft is then no longer movable along its longitudinal axis.

In several embodiments, the radial head implant comprises surfaces for the secondary anchoring on the shaft and on the threaded anchor. After an ingrowth, the shaft is no longer movable along its longitudinal axis.

In several embodiments, at least two, preferably all, parts of the radial head implant are made of the same material.

In several embodiments, one, several or all parts of the radial head implant consist of a cobalt-chromium alloy (CoCr).

In several embodiments, one, several or all parts of the radial head implant are made of titanium, particularly of titanium grade 5.

In several embodiments, the head of the radial head implant is wholly or partially made of pyrocarbon and/or ceramic.

In several embodiments, the head is made of CoCr, the threaded anchor is made of titanium (particularly of titanium grade 5), and the shaft is made of CoCr or titanium (in particular titanium grade 5).

In several embodiments, the head is partially or wholly made of pyrocarbon, wherein shaft and threaded anchor are made of CoCr.

In several embodiments, the head is partially or wholly made of ceramic, wherein the shaft and/or the threaded anchor are preferably made of titanium (in particular titanium grade 5).

For use with a radial head implant for the capitulum humeri, a head is used in several embodiments, said head comprising or consisting of a plastic (in particular polyethylene, in particular highly cross-linked polyethylene, in particular with vitamin E).

By using the same materials for several or all parts of the implant, the risk of galvanic corrosion and cold welding can be reduced or avoided.

In several embodiments, the radial head implant according to the present invention, the head, the threaded anchor, the shaft, the washer, the sawing template, the head-applying tool, the shaft-implanting tool, the ratchet, the bone processing tool, the kit and/or the method comprise one or several of the aforementioned and/or following advantages.

In several embodiments, the articulation surfaces of the radial head with the capitulum humeri and/or of the proximal radioulnar joint may be restored by the present invention. In this, pain may be reduced or permanently eliminated and/or mobility may be (again) rendered possible. By the present invention, in some cases, a primary and/or secondary stability, long service life and/or little or no abrasion may be achieved and/or undesired reactions between tissue and implant may be avoided. The present invention may often achieve the proper joint tension and contribute to the minimizing or preventing luxations and subluxations etc. Preferably, the invention is biocompatible and the biomechanics are met.

In several cases, the exact fitting of the sawing template, in particular by the size of the template with respect to the joint space, the capitulum, the radial head and/or the medial depth of the trochlea, and by the exact axial alignment with respect to radial shaft, the plate may serve as guide of the cutting plane in the xy-plane. By the openings (L/R) this cutting plane may be temporarily fixed, for the duration of the resection, to the distal end of the humerus (capitulum humeri) using a Kirschner-wire or the like. The cutting plane at the resected radial shaft thus comprises, with respect to the capitulum humeri and the rest of the anatomy, the optimal three-dimensional alignment and has the corresponding distance between capitulum humeri and the resected radial shaft by selecting the correct size also by the size harmonization for each implant (radial head). This means that thereby the radial head implant selected and used in the subsequent course of surgery is anatomically aligned and has the correct distance to the capitulum (no over-/under-stuffing) and the articulation to the radioulnar joint is established.

In several embodiments, one or several of the following are anatomically usable on both sides: head, threaded anchor, shaft, washer, radial head implant, sawing template, head-applying tool, shaft-implanting tool, ratchet, bone processing tool.

In the following, the present invention is described based on preferred embodiments thereof with reference to the accompanying drawings. However, the present invention is not to be limited to these embodiments. The following applies in the figures:

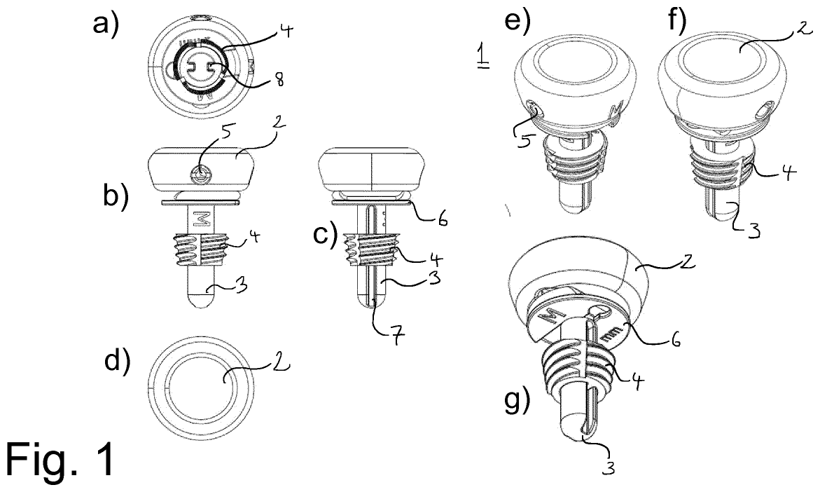

FIG. 1 shows the radial head implant according to the present invention;

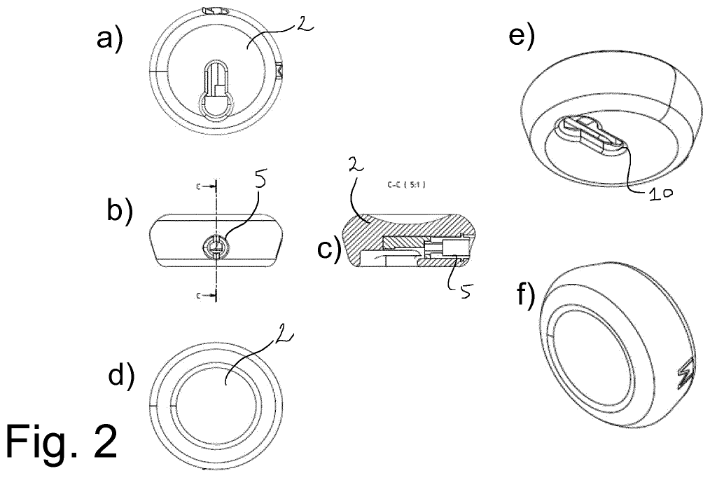

FIG. 2 shows the head according to the present invention for a radial head implant;

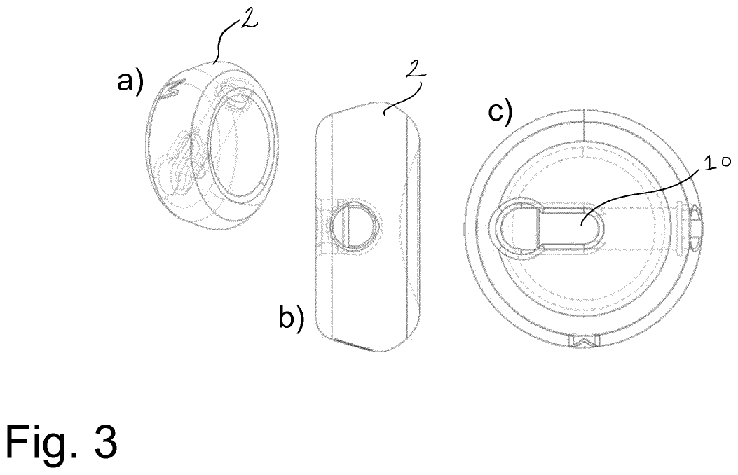

FIG. 3 shows the head according to the present invention in three further views;

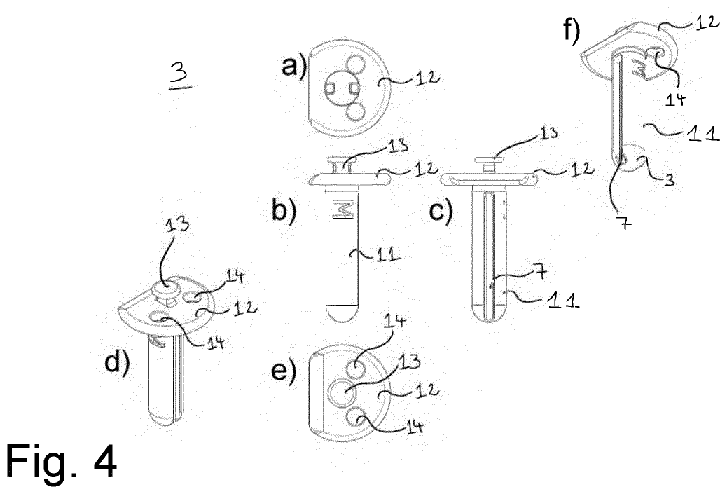

FIG. 4 shows the shaft according to the present invention;

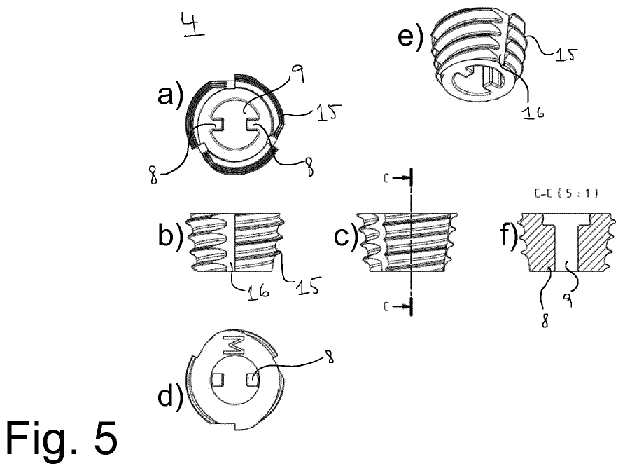

FIG. 5 shows the threaded anchor according to the present invention;

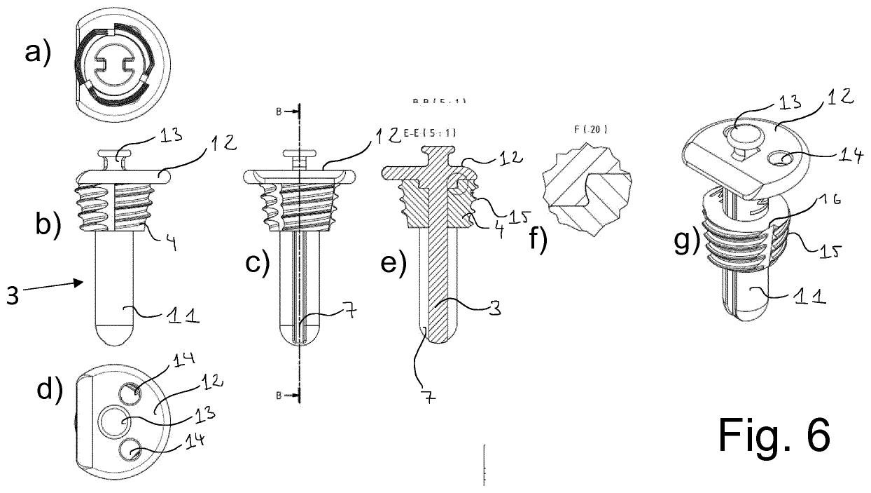

FIG. 6 shows the threaded anchor in which a shaft is inserted;

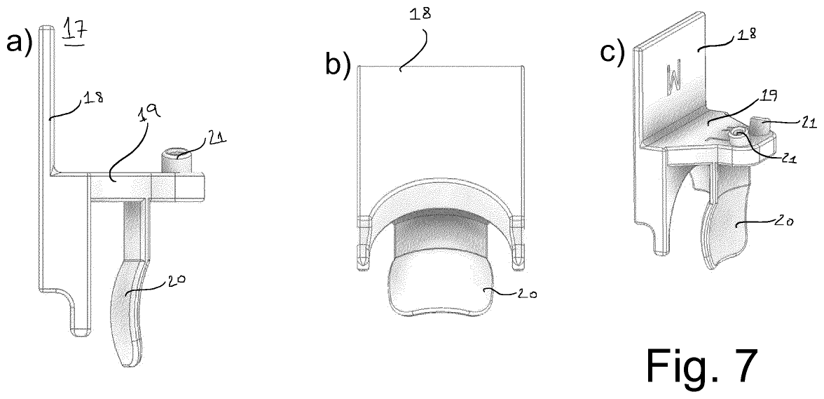

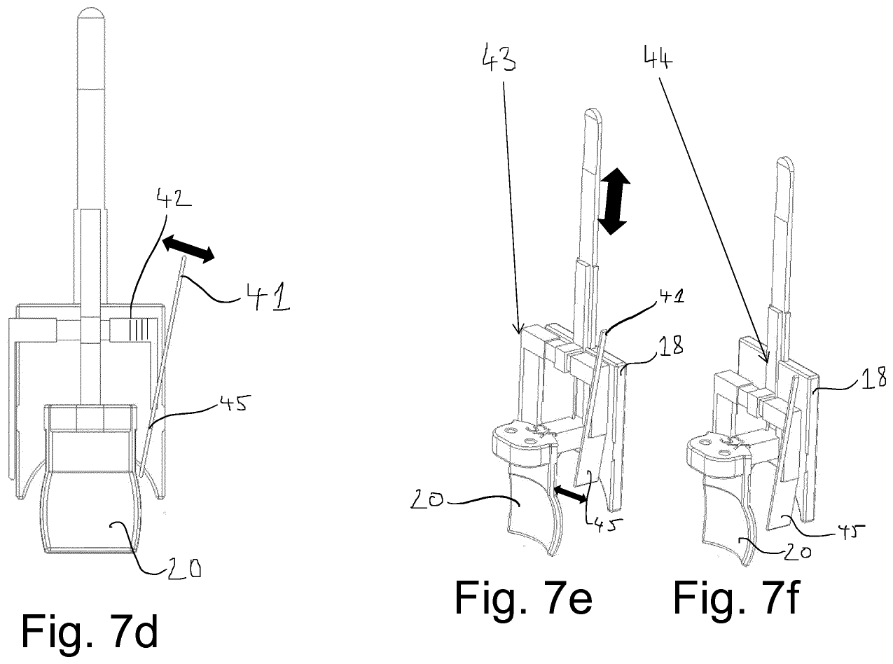

FIG. 7 shows the sawing template according to the present invention and a size gauge;

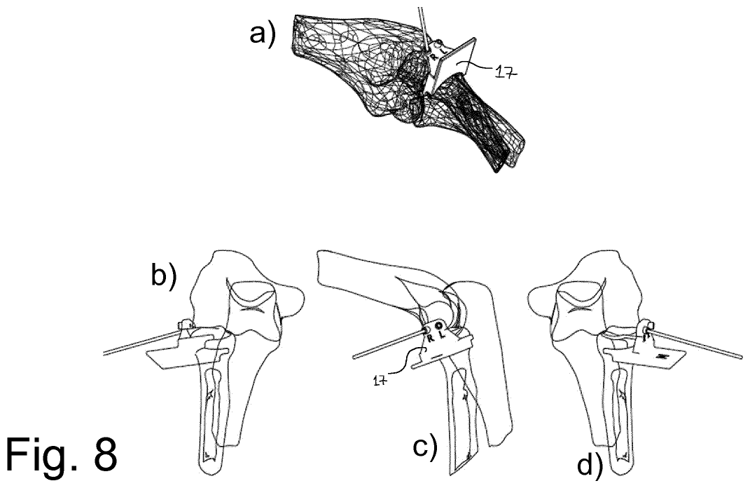

FIG. 8 shows the sawing template being inserted into a joint space;

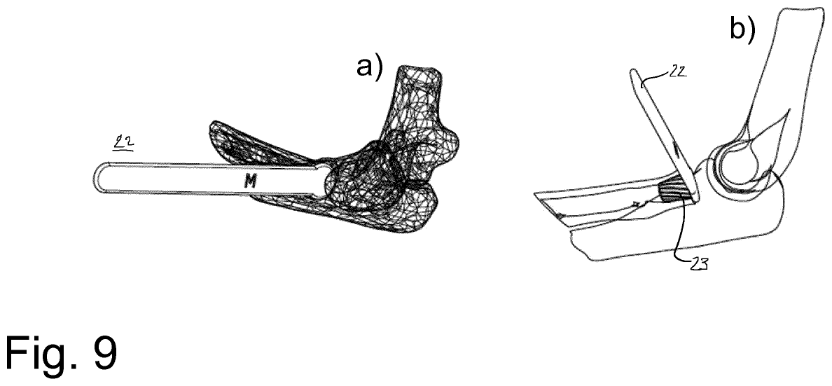

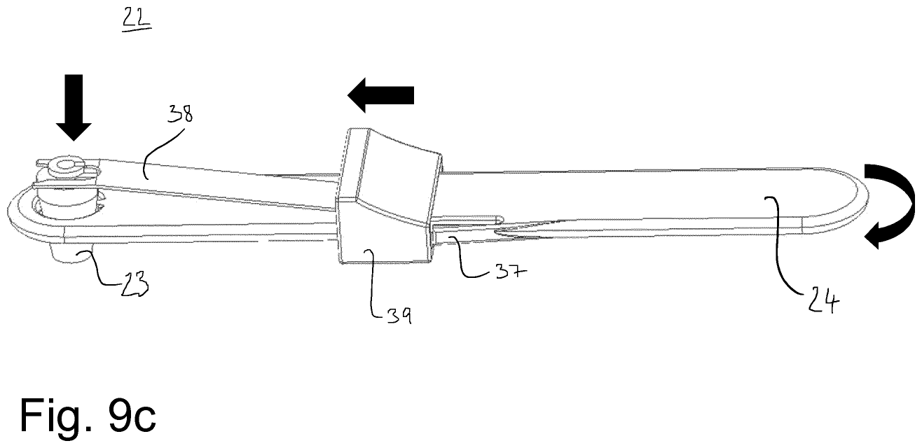

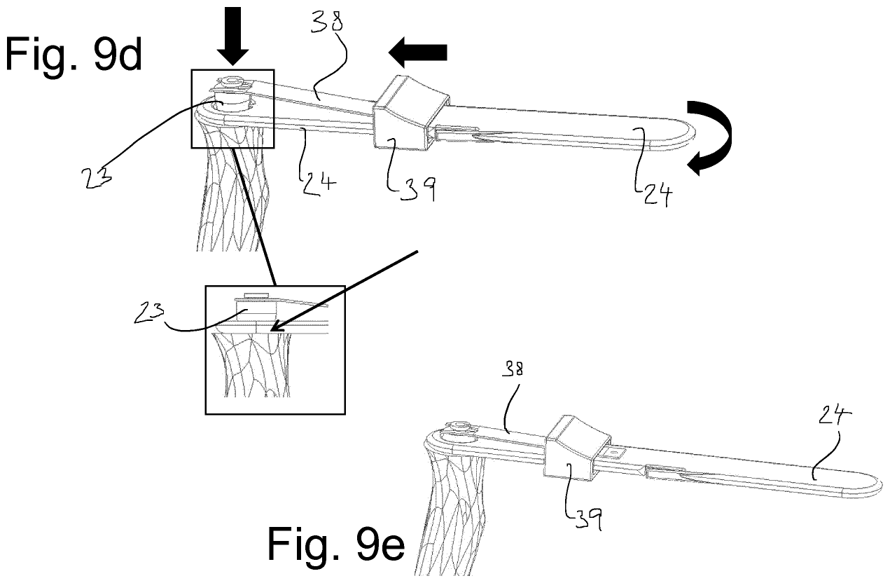

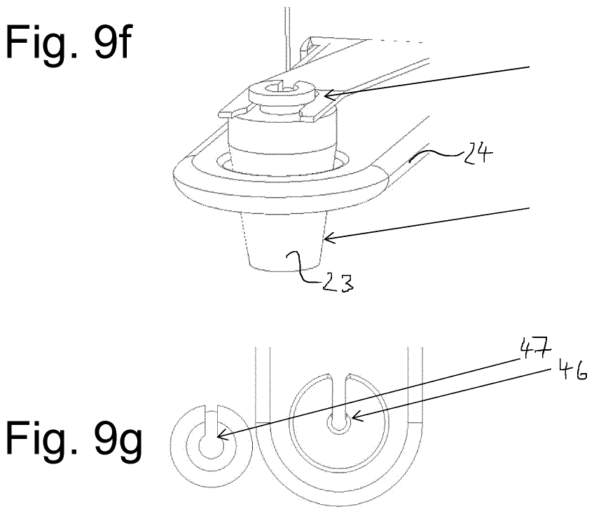

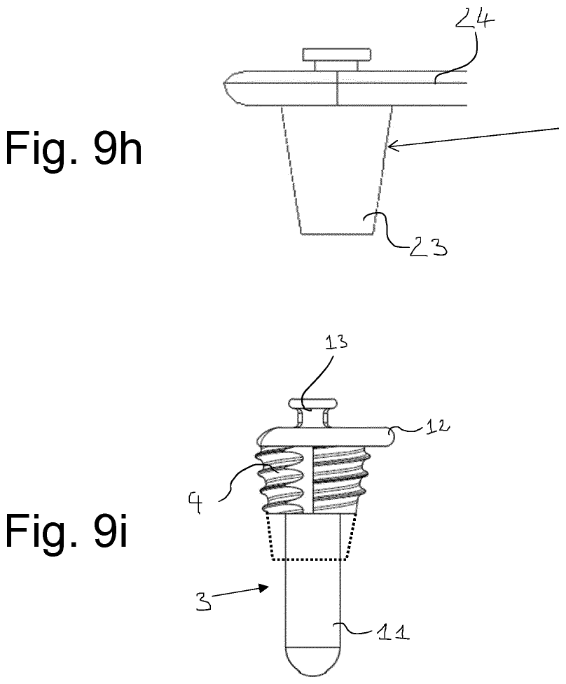

FIG. 9 shows the bone processing tool according to the present invention;

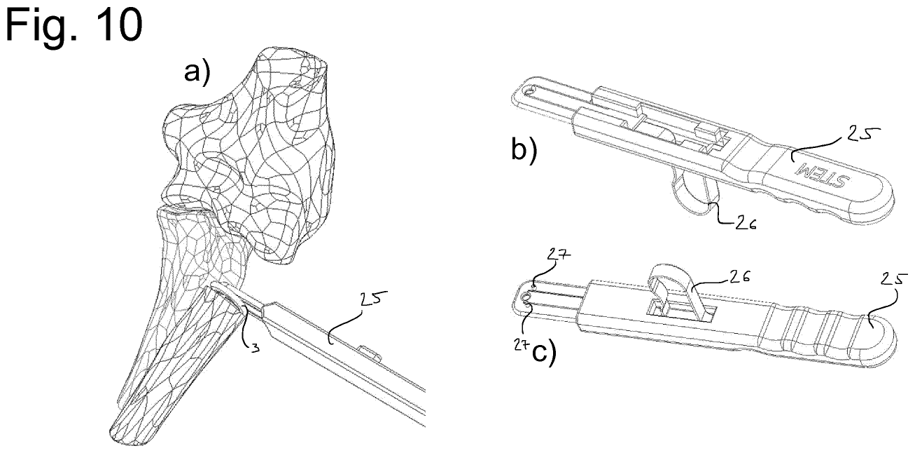

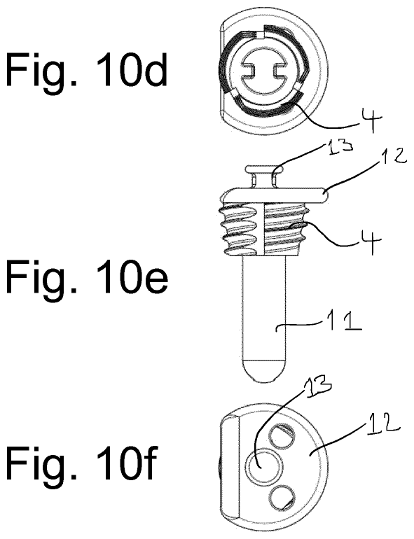

FIG. 10 shows the shaft-implanting tool according to the present invention and the shaft with applied threaded anchor;

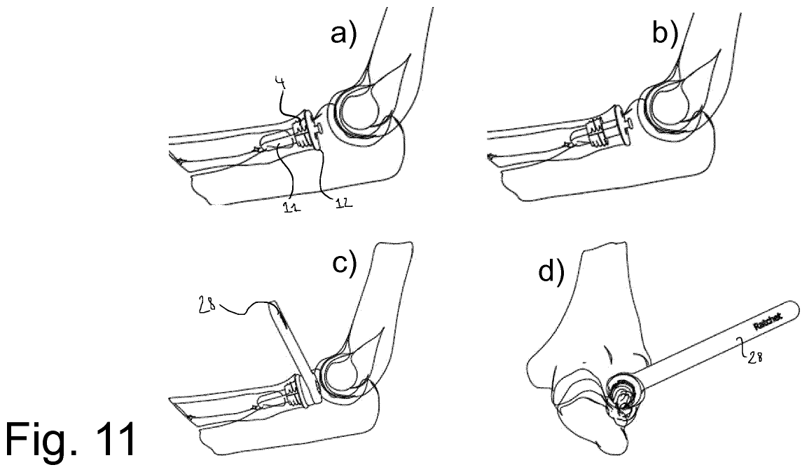

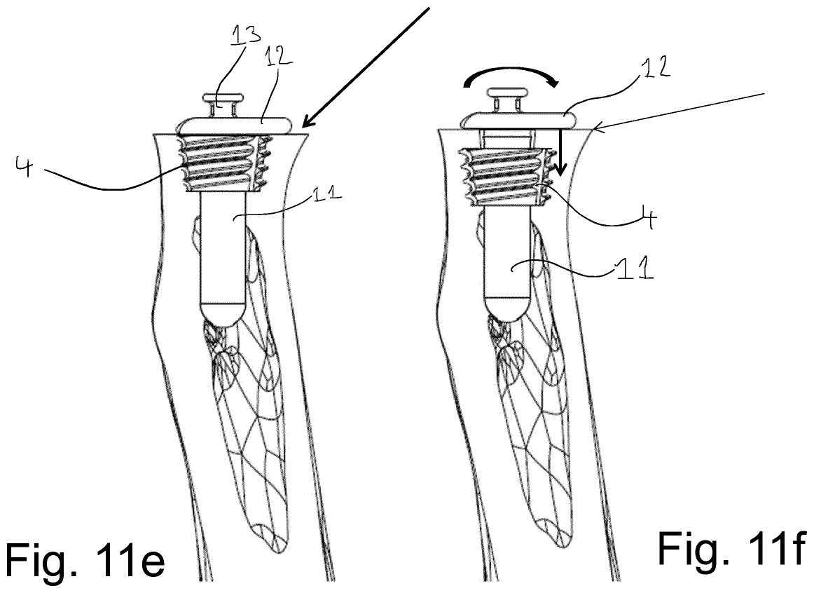

FIG. 11 shows the ratchet according to the present invention and the shaft with threaded anchor being inserted into the bone;

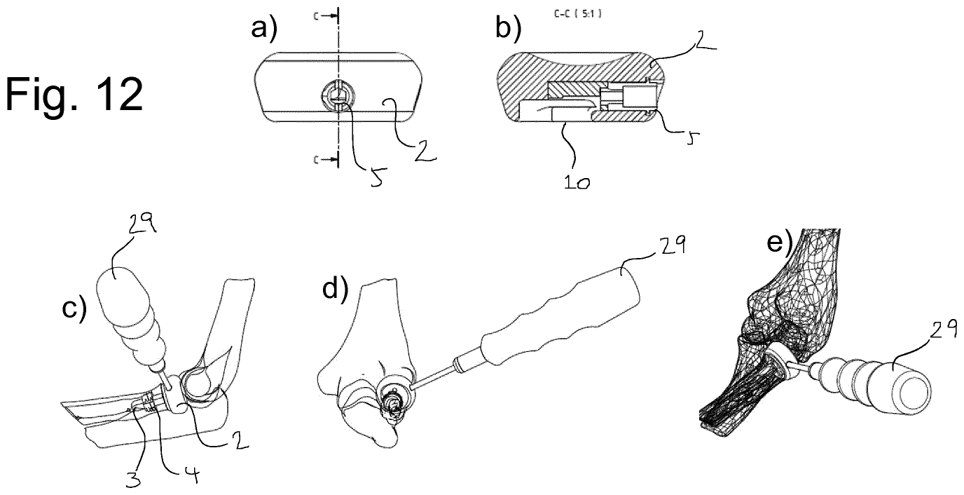

FIG. 12 shows the head-applying tool according to the present invention;

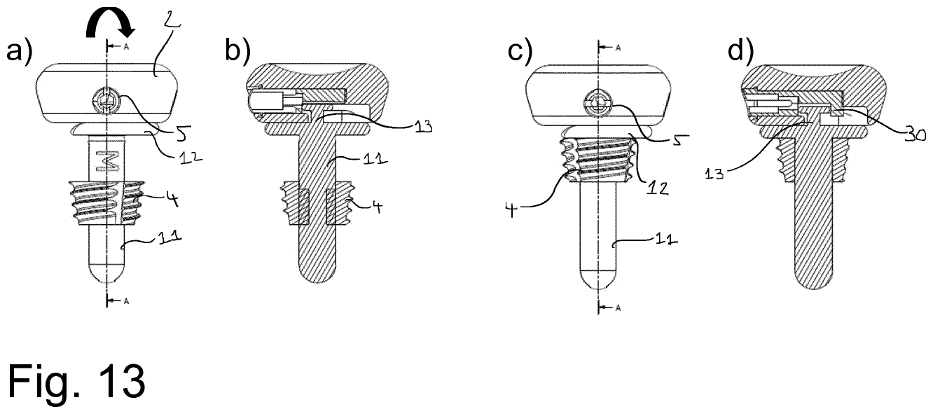

FIG. 13 shows the radial head implant with its interlocking element in two different rotation positions;

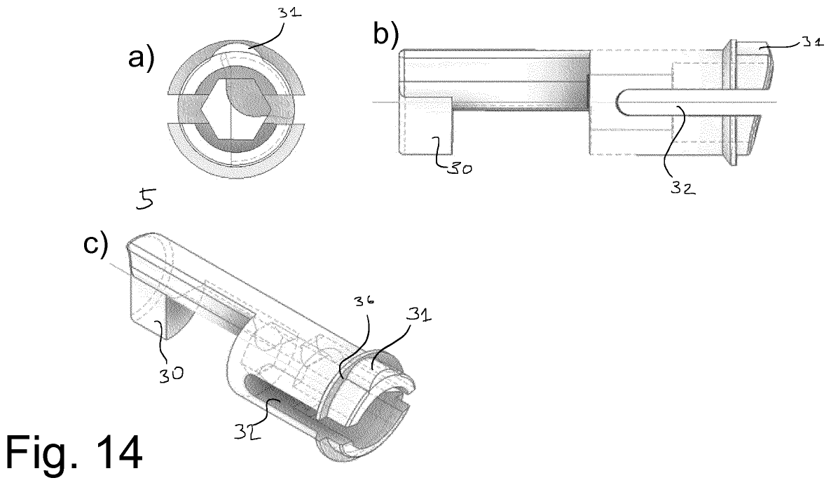

FIG. 14 shows the interlocking element of the head;

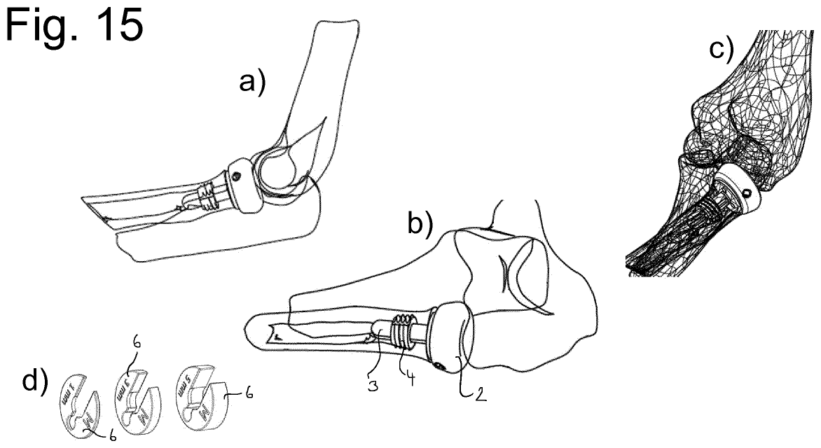

FIG. 15 shows the radial head implant, which is inserted into a radius and the washer according to the present invention; and

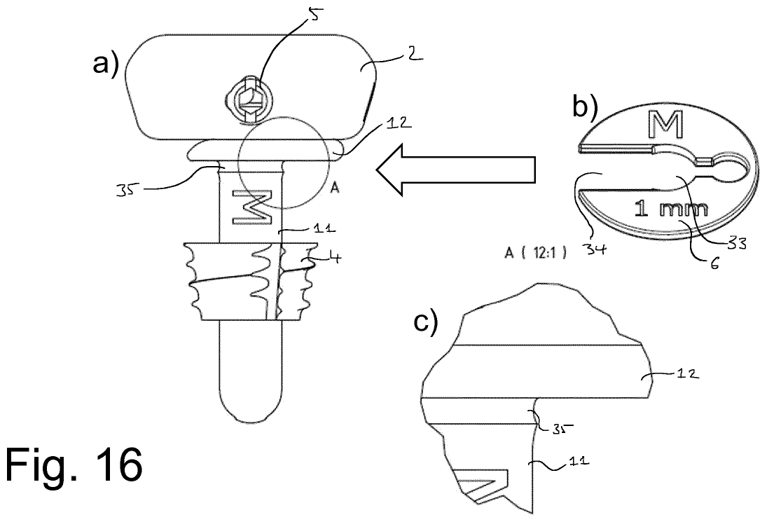

FIG. 16 shows the radial head implant with the washer in a further illustration.

FIG. 1 shows a radial head implant 1 with a head 2, a shaft 3 and a threaded anchor 4. FIG. 1a and FIG. 1d show views from below or from above, respectively. In FIG. 1b and FIG. 1c side views are shown and FIG. 1e, FIG. 1f and FIG. 1g show perspective views. In the side view of FIG. 1b, an interlocking element 5 can also be seen as well as the optional reference “M” which herein indicates the size of the implant. In FIG. 1g, an optional washer 6 is also seen which is put onto the shaft 3. In FIG. 1c, FIG. 1e and FIG. 1f, one of two (could be more than two) optional grooves 7 in the shaft 3 can be seen, which cooperates for example with two noses 8 in a through-opening 9 (see FIG. 5f) of the threaded anchor 4 and which results in a linear guide allowing an axial shifting of the shaft 3 in the threaded anchor 4. The grooves 7 and the noses 8 also allow a transmission of torque from the shaft 3 to the threaded anchor 4, which may be useful when screwing the radial head implant 1 or its threaded anchor 4 into a radial bone.

Groove(s) 7 and nose(s) 8 may alternatively be interchanged such that the groove(s) 7 is/are not provided on the shaft 3 rather on the threaded anchor 4, and vice versa.

There may be provided other elements instead of the groove(s) 7 and the nose(s) 8 for establishing a form-fit and/or force-fit connection between shaft 3 and threaded anchor 4.

FIG. 2 shows the head 2 of the radial head implant 1 in a view from below (FIG. 2a), from the side (FIG. 2b) and from above (FIG. 2d).

In FIG. 2e and FIG. 2f there is illustrated a perspective view of the head 2 from obliquely below or obliquely above, respectively. FIG. 2c shows a cut through a longitudinal axis of the interlocking element 5.

In particular, FIG. 2a and FIG. 2e clearly show an opening 10 in the head 2.

A first portion of the opening 10 comprises a larger diameter, and a second portion comprises a smaller diameter or smaller width (e.g. less than the diameter of the first portion). The portion of the opening 10 with the larger diameter comprises here a circular limitation. A subsequent rectangle (which herein again extends into a smaller circle or semicircle) comprises a smaller diameter or a smaller width.

First portion and second portion may together optionally have the shape of a keyhole.

In FIG. 3a to FIG. 3c there is again shown the head 2 with the opening 10 and the opening for inserting the interlocking element 5.

FIG. 4 shows a plurality of illustrations of the shaft 3 with a lower shaft part 11 and an upper shaft part 12 and the optional groove 7. A holding pin 13 of the upper shaft part 12 shows herein a wider, upper portion, in relation to the bottom portion, with an optional circular surface or base area. The bottom portion of the holding pin 13 lies between the upper portion and the proximal surface of the upper shaft part 12. The bottom portion of the holding pin 13 comprises herein purely optionally an angular and not a round cross section.

Two openings 14 are introduced in the upper shaft part 12. These can be used to transmit a torque from a tool to the shaft 3, for example, to screw the shaft 3 with its threaded anchor 4 into the radius.

The cut through the upper shaft part 12 in a plane perpendicular to the longitudinal axis of the lower shaft part 11 results in the example of FIG. 4 in a form which is defined through a (part-)arc (alternatively, a non-circular, for instance an oval (part) arc, the ends of which are connected by a straight line. In other words, the section has the shape of a circular section. By the shape of the upper shaft part 12, thus flattened, inserting the shaft 3 into the radius is facilitated in particular with minimally invasive surgery. The shaft 3 may thus be advantageously inserted into the radius also in case of a narrow operative access.

Instead of the two openings 14, a plurality of openings 14 may be provided. Alternatively, an opening 14 and a stop or the like would be sufficient to ensure the desired form-fit and/or force-fit connection for the tool

In FIG. 5, the threaded anchor 4 is shown from above (FIG. 5d), from below (FIG. 5a) and from the side (FIGS. 5b and 5c). FIG. 5f shows a sectional view, and in FIG. 5e the threaded anchor 4 is shown in a perspective view obliquely from below. The outer shape of the threaded anchor 4 corresponds substantially to a truncated cone, on which a thread is applied.

The thread 15 of the threaded anchor 4 is self-tapping, in particular also in left-hand rotation, to facilitate the removal of the radial head implant.

The thread 15 optionally comprises interruptions 16 at various (here: purely exemplary three) positions of the circumference in order to remove the bone material released during the threading and/or to break the chip.

In FIG. 5, the through opening 9 of the threaded anchor 4 is also illustrated. The noses 8 optionally present in the through-opening 9 for engaging in the grooves 7 of the lower shaft part 11 are also seen here. The noses 8 allow an axial shifting of the shaft 3 relative to the threaded anchor 4. In addition, a torque may be transmitted from the shaft 3 to the threaded anchor 4 via the grooves 7 and the noses 8.

FIG. 6 shows illustrations of the shaft 3 with applied or attached threaded anchor 4 from above (FIG. 6d), from below (FIG. 6a), from the side (FIGS. 6b and 6c), in section through the longitudinal axis (FIG. 6e), with a detail illustration (FIG. 6f) and in a perspective illustration obliquely from above (FIG. 6g).

The threaded anchor 4 is, in FIG. 6b to FIG. 6f, optionally releasably and securely jammed or snapped on the shaft 3. For this purpose, the portions of the lower shaft part 11 shown in FIG. 6e and enlarged in FIG. 6f engage in the proximal portion of the threaded anchor 4 at the transition to the upper shaft part 12. Attaching the threaded anchor 4 on the shaft 3 in a releasable and secure (against loss) manner prevents when inserting the shaft 3, an undesired falling out of the threaded anchor 4 which could potentially prolong or extend an operation or could even cause the threaded anchor 4 to become non-sterile. This is prevented by the shown releasable securing element.

In FIG. 7, the sawing template 17 according to the present invention is shown from the side (FIG. 7a), distally (FIG. 7b) and in perspective view from obliquely above (FIG. 7c).

The sawing template 17 comprises a plate 18 and a tongue 20 mounted via a connecting element 19 parallel to the sawing template 17. In addition, in FIG. 7a and FIG. 7c, through-openings 21 are shown which extend or continue to the outside in a short hollow cylinder. The through openings 21 serve for inserting wires with which the sawing template 17 may be fixed to the humerus. In this, only one through-opening 21 is used when operating the left arm and only the other through-opening 21 is used when operating the right arm.

To avoid confusion between them, the through-openings 21 are optionally marked on the connecting element 19, for instance with R and L.

The plate 18 has towards the radial head a preferably circular arc-shaped limitation, so that the plate 18 can support itself with the largest possible surface on the radial head. The tongue 20 optionally comprises a convexity in the direction of the plate 18, which serves for lying better on the concave joint surface of the radial head.

The sawing template 17 is inserted before the implantation together with its tongue 20 into the joint space, wherein the circular arc-shaped lower limitation of the sawing template 17 lies or rests laterally on the radial head. Subsequently, it is evaluated whether the sawing template 17 fits the radial head sufficiently accurately or whether another sawing template 17 with a different size should be used. This may be assessed haptically or visually. In this context, the sawing template 17 is used for determining the size of the radial head. When the correct size has been found, then the corresponding sawing template 17 may be inserted into the joint space and placed on the radius and fixed with a wire, as shown in FIG. 8a to FIG. 8d. Thereafter, the distal surface of the plate 18 may be used to guide for instance an oscillating saw to resect the radial head.

In several embodiments (FIG. 7d to FIG. 7f), the sawing template 17 may be used in conjunction with a size gauge 43. In this, the size gauge 43, which is guided in the plate 18 of the sawing template 17, may be put on the radial head for example with the aid of a slide 44 of the size gauge 43. In this, an arm 45 or the size gauge 43 is deflected by contact with the lateral edge of the radial head, so that an indicator 41 connected to the arm 45 is deflected. The deflection of the indicator 41 may be read on a scale 42 applied on the size gauge 43. In this, a value of the scale 42 corresponds to a specific size of a radial head, so that corresponding implant sizes and tools matching thereto in terms of size can be selected on the basis of the scale value.

The size of the resected radial head may be checked with a template (not shown) which has circular recesses of different diameters.

After the resection, a bone processing tool 22 with a rasping element 23 may be introduced into the medullary cavity of the radius (see FIG. 9a and FIG. 9b). By pivoting a stalk 24 of the bone processing tool 22, the medullary cavity is expanded by rasping. For this purpose, the surface of the rasping element 23 comprises for example notches or teeth.

In one embodiment (see FIG. 9c) of the bone processing tool 22 with stalk 24, this comprises a pusher 39 which may be shifted along a leaf spring 38. The rasping element 23 is fastened at the leaf spring 38. By shifting the pusher 39, the rasping element 23 may be pushed through the stalk 24. The pusher 39 is optionally equipped with a securing element, herein optionally designed as snap-in tongue which prevents the pusher 39 from slipping off the rasping element 23 away from the stalk 24.

The stalk 24 of the bone processing tool 22 may be designed as a ratchet.

The rasping element 23 may be designed to be interchangeable or replaceable, so that e.g. rasping elements 23 having different sizes may be used with the bone processing tool 22.

In FIG. 9d and FIG. 9e there is shown how to use the bone processing tool 22. The bone processing tool 22 lies thereby flatly with its stalk 24 on the resection plane of the radius. The axis of rotation of the rasping element 23 is thereby perpendicular to the resection plane. By rotational movements of the bone processing tool 22, the conical rasping element 23 being sprung by the leaf spring 38 cuts into the medullary cavity of the radius.

FIG. 9f and FIG. 9g show detailed views of the bone processing tool 22. In this, it can be seen that there is optionally a securing element 46 integrated in the leaf spring 38 which securing element 46 engages in the rasping element 23 and moreover serves for operating the rasping element 23. The securing element 46 of the leaf spring 38 is in FIG. 9g optionally designed as a tongue which extends in a narrow web leading away from the remaining leaf spring 38 and widening itself to a circular shape at its free end. The tongue is inserted into the rasping element 23 for the use of the bone processing tool 22 and transmits torques of the stalk 24 to the rasping element 23. For this purpose, the rasping element 23 optionally comprises a depression 47 corresponding to the securing element 46, in particular of the tongue.

The rasping element 23 is preferably designed conically and cuts, into a desired, size-dependent depth, into the radius by rotational movements of the stalk 24 and by the contact pressure of leaf spring 38 and pusher 39.

In this, the rasping element 23 is preferably matched to the dimensions of the threaded anchor 4, as shown in FIG. 9h and FIG. 9i.

In several embodiments, the rasping element 23 may comprise two or more steps. The conical rasping element 23 creates space in the radius for the threaded anchor 4 and for the shaft 3 by the removal of bone material using the bone processing tool 22. In addition, the rasping element 23 cuts free the space in the radius up to the base of the thread of the threaded anchor 4 thus allowing screwing in the threaded anchor 4 (see the broken line in FIG. 9i).

After processing the bone using the bone processing tool 22, the bone is ready for the insertion of shaft 3 with the optionally captively-secured attached threaded anchor 4.

The insertion is performed using a shaft-implanting tool 25 which is shown in FIG. 10a to FIG. 10c. The shaft implantation tool 25 comprises an optional elastic element 26 which serves for fixing or fastening the holding pin 13 onto the bone processing tool 22. The bone processing tool 22 also comprises two or more pins 27 which may be inserted into the openings 14 of the upper shaft part 12 to securely and precisely insert the shaft 3 into the radius. The situation after the insertion of the shaft 3, wherein the shaft 3 is still fastened at the bone processing tool 22, is shown in FIG. 10a.

In FIG. 10d to FIG. 10f it can be seen that the upper shaft part 12 viewed from above (FIG. 10f) and viewed from below (FIG. 10d), is laterally optionally flattened, e.g. in the sense of a circular section. In addition, it can be seen in FIG. 10e that the upper shaft part 12 can be beveled in proximal direction on its flat side, which may additionally facilitate a minimally invasive tipping of the shaft 3 into the radius.

The inserted shaft 3 having a threaded anchor 4 is shown in FIG. 11a, the threaded anchor lies herein still on the upper shaft part 12 and is not yet screwed into the bone. A ratchet 28 may then be applied on the upper shaft part 12, as shown in FIG. 11c and FIG. 11d. By rotating the ratchet 28, a torque may be transmitted to the threaded anchor 4 via the shaft 3 rotating due to the ratchet 28 so that the threaded anchor 4 is may be screwed deeper into the radius. However, since the shaft 3 is axially freely movable in the threaded anchor 4 (by engagement or guidance between groove 7 and noses 8), the position of the shaft 3 does not necessarily have to change due to screwing in the threaded anchor 4.

In FIG. 11e and FIG. 11f, it is illustrated that the upper shaft part 12 is optionally designed to rest on or abut the resection surface (indicated by arrows in FIG. 11e and 11f) of the radius and thus to provide a desired axis of rotation of the lower shaft part 11 with threaded anchor 4 or to provide at least the inclination of the rotation axis.

FIG. 11e shows the shaft 3 with threaded anchor 4 in an initial position after insertion. FIG. 11f shows that the threaded anchor 4—due to the rotation of the shaft 3, is screwed distally—i.e. into the radius.

In FIG. 12, a head-applying tool 29 is shown with the head 2 is attached to the inserted shaft 3 and/or with which the head 2 may interlock with the shaft 3 after being attached thereto or thereon. For this purpose, the head-applying tool 29 engages at the interlocking element 5.

In the example shown in FIG. 12, the interlocking element 5 optionally has a hexagon socket in which the head-applying tool 29 engages. Other geometrical forms such as the hexagon socket are also encompassed by the present invention.

The head-applying tool 29 is moved such that the holding pin 13 is introduced into the opening 10 of the head 2 or the head 2 is pushed with its opening 10 on the holding pin 13, in particular in an axial direction of the holding pin 13.

Thereafter, the head 2 is further moved, in particular in a radial direction, until the holding pin 13 is present in the narrower portion of the opening 10, thus, the holding pin 13 cannot be axially removed or pulled from the head 2.

In this position, the holding pin 13 is interlocked with the head 2. This is done by rotating the interlocking element 5 from an open to a closed rotation position using the head-applying tool 29.

In the closed rotational position, e.g. a latch 30 prevents that the holding pin 13 slips back into the wider portion of the opening 10. Thus, the head 2 is fastened relative to the upper shaft part 12 and thus also relative to the shaft 3.

In FIG. 13, the two rotational positions of the interlocking elements 5 can be clearly seen, wherein the position of the threaded anchor 4 relative to the shaft 3 needs not to be observed.

FIG. 13a and FIG. 13b show the open rotational position, while FIG. 13c and FIG. 13d show the closed rotational position.

In FIG. 13d, there is shown in a section that the latch 30 is in the way of the holding pin 13 or it limits its freedom of movement. Hence, this results in a form-fit connection, which prevents the holding pin 13 from slipping from the narrower into the wider portion of the opening 10 (here from left to right). Due to said limitation, the holding pin 13 cannot be removed from the head 2 anymore. In the open rotation position, the latch 30 is rotated upwards and releases the wider portion of the opening 10 (FIG. 13a and FIG. 13b). This makes it possible to introduce the holding pin 13 into the head 2 through the wider portion of the opening 10 or to remove it therefrom.

FIG. 14 shows the interlocking element 5. It is clearly seen that the interlocking element 5 has an optional hexagon socket on which the head-applying tool 29 may be applied in order to transmit a torque to the interlocking element 5.

An optional detent element 31 of the interlocking element 5 may cooperate with a corresponding depression of the head, such that the interlocking element 5 may engage in an open and/or closed rotational position.

In addition, the interlocking element 5 comprises an optional securing element 36 which is latched in e.g. a groove of the head 2.

An optional slot 32 in the interlocking element 5 may provide the necessary elasticity for latching. In addition, the slot 32 may preferably be used to rotate the interlocking element 5 with a screwdriver.

FIG. 15 shows washers 6 and their use. If it is ascertained, after introducing the radial head implant 1, that the joint space is still too wide, as indicated in FIG. 15a, and FIG. 15b by a slot between radius and lower side of the head 2, then a washer 6 may introduced for correction in this slot between upper shaft part 12 and radius. The washers 6 may be provided in different thicknesses, for example, with a thickness of 1, 3 and 5 mm.

In FIG. 16a, a radial head implant 1 is shown which optionally comprises, on the lower shaft part 11, e.g. a circumferential groove 35 for pushing the washer 6 on the lower shaft part 11. The groove 35 may prevent the washer 6 from slipping axially.

In FIG. 16b, a washer 6 is illustrated in detail.

The washer 6 comprises a central hole 33, which optionally continues into a slot 34 which extends until the edge of the washer 6. In this, the slot 34 is preferably slightly narrower than a diameter of the lower shaft part 11 such that the washer 6 must be slightly (pre-)tensioned for being pushed onto the lower shaft part 11.

In order to improve the elasticity of the washer 6, as shown in FIG. 16b, material may be removed from the portion of the washer 6 which lies on the side of the hole 33 away from or opposite of the slot 34. Herein, there is shown an opening which adjoins or extends into the hole 33. When the lower shaft part 11 has reached the central hole 33 during insertion, the washer 6 relaxes. Thus, the washer 6 is prevented from slipping again off the lower shaft part 11 after the completed insertion. This may be supported by the interlocking, detention or clamping which may be seen in FIG. 16b.

### LIST OF REFERENCE NUMERALS

1 radial head implant
2 head
3 shaft
4 threaded anchor
5 interlocking element
6 washer
7 groove
8 nose
9 through-opening of the threaded anchor
10 head opening or opening of or in the head
11 lower shaft part
12 upper shaft part
13 holding pin
14 bores or openings in the upper shaft part
15 thread of the threaded anchor
16 thread interruptions or threadless or thread-free section
17 sawing template
18 sawing template plate
19 connecting element of the sawing template
20 sawing template tongue
21 through-opening of sawing template
22 bone processing tool
23 rasping element
24 stalk of the bone processing tool
25 shaft-implanting tool
26 elastic element of the shaft-implanting tool
27 pins of the bone processing tool
28 ratchet
29 head-applying tool
30 latch of the interlocking element
31 detent element of the interlocking element
32 interlocking element slot
33 washer hole
34 washer slot
35 closed groove of the lower shaft part
36 securing element of the interlocking element
37 securing element of the pusher of the bone processing tool
38 leaf spring of the bone processing tool
39 pusher of the bone processing tool
41 size gauge indicator
42 size gauge scale
43 size gauge
44 size gauge slide
45 size gauge arm
46 securing element of the rasping element of the bone processing tool
47 rasping element depression

1. The radial head implant according to claim 15, wherein the head comprises an interlocking element for fastening the head.

2. The radial head implant according to claim 1, wherein the interlocking element comprises a latch, a securing element and/or a detent element for at least one rotation position.

3. The radial head implant according to claim 15, wherein the head is designed concavely on an upper side for articulation with the capitulum humeri.

4. The radial head implant according to claim 15, wherein the head comprises an opening at a bottom or lower side with at least two different dimensions in one extension direction.

5. The radial head implant according to claim 15, wherein the threaded anchor comprises a self-tapping thread and a central through-opening.

6. The radial head implant according to claim 15, wherein the threaded anchor comprises a body that tapers in distal direction.

7. The radial head implant according to claim 15, wherein the shaft has an upper shaft part and a lower shaft part, wherein the upper shaft part is designed to be fastened to a head and the lower shaft part is designed to be inserted into a radius.

8. The radial head implant according to claim 7, wherein the lower shaft part may be inserted into a central through-opening of a threaded anchor, in particular according to claim 5 and/or claim 6.

9. The radial head implant according to claim 7, wherein the lower shaft part comprises at least one groove.

10. The shaft according to claim 7, wherein the upper shaft part comprises a holding pin for cooperation or interaction with the head.

11. The radial head implant according to claim 7, wherein the upper shaft part, measured from a longitudinal axis of the lower shaft part, comprises an extension in a first direction which differs from an extension in a second direction.

12. The radial head implant according to claim 7, wherein the upper shaft part comprises a base surface which is defined or limited by a circular arc and a straight line.

13. The radial head implant according to claim 7, wherein the upper shaft part comprises at least one bore for transmitting a torque.

14. A radial head implant according to claim 15, comprising a washer to be inserted between shaft and bone.

15. A radial head implant with a head, a threaded and a shaft.

16. A sawing template with a plate and a tongue mounted parallel to the plate, the tongue having a convexity towards the plate.

17. (canceled)

18. A shaft-implanting tool for inserting the shaft with the threaded anchor into the medullary cavity of a radius.

19. (canceled)

20. A bone processing tool with a stalk and a rasping element arranged laterally.

21. A kit with a radial head implant according to claim 15 and comprising at least one of the following aids:
a sawing template with a plate and a tongue mounted parallel to the plate, the tongue having a convexity towards the plate;
a head-applying tool for receiving the head and for fastening the head on a shaft being inserted in a radius;
a shaft-implanting tool for inserting the shaft with the threaded anchor into the medullary cavity of a radius;
a ratchet for screwing the shaft together with the threaded anchor into the radius; and
a bone processing tool with a stalk and a rasping element arranged laterally.

22. A method for inserting a radial head implant encompassing the steps:
positioning and/or aligning a sawing template (17) according to claim 16 at a radial head, at a capitulum humeri and/or at its joint space and positioning and/or aligning the sawing template along a radial shaft;
determining the size of at least one of the radial shaft and of the radial head;
resecting the radial head along a side of the plate or guide of the sawing template, the side being distal to the radial head;
extending the medullary cavity of the radius using a bone processing tool with a stalk and a rasping element arranged laterally;
inserting a shaft with a threaded anchor into the medullary cavity of the radius using a shaft-implanting tool for inserting the shaft with the threaded anchor into the medullary cavity of a radius;
screwing the shaft with threaded anchor into the radius using a ratchet for screwing the shaft together with the threaded anchor into the radius;
placing the head on the shaft using a head-applying tool for receiving the head and for fastening the head on a shaft being inserted in a radius; and
interlocking the interlocking element of the head using the head-applying tool.
